(19) United States
(12) Patent Application Publication
(10) Pub. No.: US 2010/0197007 A1
(43) Pub. Date: Aug. 5, 2010

(54) Method for Culturing Mammalian Stem Cells

(75) Inventors: Michel Cailleret, Boissise le Roi (FR); Julien Come, Ste Genevieve des Bois (FR); Marc Peschanski, Creteil (FR)

Correspondence Address: MCANDREWS HELD & MALLOY, LTD, 500 WEST MADISON STREET, SUITE 3400, CHICAGO, IL, 60661, US

(73) Assignee: Inserm(Institut National de La Sante et de la Recherche Medicale), Pari Cedex l3 (FR)

(21) Appl. No.: 12/671,105

(22) PCT Filed: Jul. 23, 2008
(86) PCT No.: PCT/EP2008/059644

§ 371 Date: Jan. 28, 2010

(30) Foreign Application Priority Data
Jul. 31, 2007 (EP) 07113509.9

(51) Int. Cl. C12N 5/0735; C12N 5/071; C12M 1/00

(52) U.S. Cl. 435/325; 435/289.1

(57) ABSTRACT

The invention relates to a method for culturing mammalian stem cells, in particular embryonic stem cells comprising the following steps: a) providing a perfused bioreactor (1) comprising a cell culture chamber (2); b) placing said mammalian stem cells within said culture chamber (2); c) providing a perfusion loop which provides fresh medium to said perfused bioreactor and removes used medium from said perfused bioreactor; d) providing a dialysis loop which comprises a reservoir of medium (3) and dialysis chamber (4); wherein the dialysis loop provides fresh medium to the perfusion loop through the dialysis chamber (4). The invention also relates to a device for culturing mammalian stem cells according to the invention.

### FIELD OF THE INVENTION

The invention relates to a method for culturing mammalian stem cells.

### BACKGROUND OF THE INVENTION

Mammalian cells are a widely used in vitro model in diagnostic and medical applications. For example, mammalian cells may be used for screening drugs, studying molecular pathways, or for the production of therapeutics drugs. Mammalian cells can also be used for cell therapy.

Mammalian stein cells are primal cells found in all mammalian organisms that retain the ability to renew themselves through mitotic cell division and can differentiate into a diverse range of specialized cell types. Embryonic stem cells (ES cells) are cultures of cells derived from the epiblast tissue of the inner cell mass of a blastocyst. A blastocyst is an early stage embryo—approximately 4 to 5 days old in humans and consisting of 50-150 cells. ES cells are pluripotent, and give rise during development to all derivatives of the three primary germ layers: ectoderm, endoderm and mesoderm. This means that they can differentiate into each of the more than 200 cell types of the adult body when given sufficient and necessary stimulation for a specific cell type.

Due to their two fundamental attributes of unlimited expansion and pluripotency, stem cells, and in particular human embryonic stem (hES) cells have gained considerable interest for use in cell replacement therapy as well as in drug discovery. ES-derived cell progeny for regenerative medicine would indeed address current acute problems of tissue shortage (see Mitjavila-Garcia et al., 2005 for a review). In parallel, the capacity to provide a practically unlimited supply of cells capable of differentiating in any cell type of interest makes ES cells a valuable tool in pharmacology (see Gorba et al., 2003 and Mc Neish et al., 2004 for reviews).

Fulfilling these promises requires, however, making full usage of both capacities of human ES cells for unlimited expansion and pluripotency. For that purpose, these two abilities have to be transcribed into technological platforms for mass cell production and controlled differentiation.

Mass cell production of mammalian stein cells, for example, ES cells or hES cells, relies on the use of bioreactors. Bioreactors typically include a housing that contains cells and nutrients maintained at bioreactor conditions that permit cell growth and/or production of secreted products. Bioreactors used for mammalian stein cell culture are well known in the art, and are commercially available from a variety of manufacturers. For example, bioreactors may include spinner flasks, roller bottles (see U.S. Pat. No. 8,866,419), hollow fibers (see U.S. Pat. No. 3,997,396), gas permeable bags (see U.S. Pat. No. 6,190,913) and porous bed reactors (see U.S. Pat. No. 5,510,262).

Conventional stirrer vessels may have the disadvantage of generating shear forces and, although manageable, these forces still damage the cells. Shear force can occur when the cells contact impeller blades (which circulate the nutrient media), the walls of the culture vessel, the fluid media, or even each other. In culture systems that bubble air, oxygen, or other gases through the media, the surface of the bubbles themselves can cause shear.

Low shear stress bioreactors have been developed for cultivating cells which are particularly sensitive to shear stress and for example for the growth of multicellular bodies as in tissue engineering. The use of low shear stress bioreactors for culturing hES cells has enabled to provide dynamic, yet mild, suspension conditions in order to control the aggregation processes of differentiating cells. For example, Gerech et al. have succeeded in obtaining mass production of embryoid bodies derived from human ES cells (hEBs) using a slow lateral turning vessel (SLTV) (Gerech et al., 2004).

However culturing mammalian stem cells in low shear stress bioreactors generally requires the renewal of large quantities of culture medium over time, especially if the cells are cultured at a high density. Mass cell production in bioreactors indeed requires very large volume of culture medium and, in parallel, increases considerably the amount of adjunct products, some of which—particularly cytokines—are extremely expensive (Itsykson et al., 2005 and Tian et al., 2004). Moreover, high density cultures suffer from an accumulation of metabolic waste products, which are deleterious to the cells.

Finally, most low shear stress bioreactors do not enable full accessibility of the closed chambers of the bioreactors without any disruption of the dynamic cell suspension. This accessibility is necessary both for external analysis and for adjunction of exogenous product, in order to provide a workable platform for implementation of any requested control and medium adaptation.

Therefore there is still an unmet need for a method for culturing mammalian stem cells, in particular ES cells, which enables long term cultures, with a reduced amount of medium and adjunct products and an efficient elimination of waste products.

### SUMMARY OF THE INVENTION

In fulfilling this need, the invention relates to a method for culturing mammalian stem cells comprising the following steps:
a) providing a perfused bioreactor comprising a cell culture chamber;
b) placing said mammalian stem cells within said culture chamber;
c) providing a perfusion loop which provides fresh medium to said perfused bioreactor and removes used medium from said perfused bioreactor;
d) providing a dialysis loop which comprises a reservoir of medium and dialysis chamber;
   wherein the dialysis loop provides fresh medium to the perfusion loop through the dialysis chamber.

The invention also relates to a device for culturing mammalian stem cells comprising:
a perfused low shear stress bioreactor with high mass transfer capacity comprising a cell culture chamber;
a perfusion loop connected to said perfused bioreactor;
a dialysis loop comprising a dialysis chamber connected to said perfusion loop;
   wherein, when in use, the perfusion loop provides fresh medium to the perfused low shear stress bioreactor and removes used medium from the perfused low shear stress bioreactor; and
   wherein, when is use, the dialysis loop provides fresh medium from a reservoir of medium to the perfusion loop through the dialysis chamber.

The invention also relates to a system comprising a device according to the invention, and mammalian stem cells placed within the culture chamber of the perfused low shear stress bioreactor.

### DETAILED DESCRIPTION

The invention relates to a method for culturing mammalian stem cells comprising the following steps:
a) providing a perfused bioreactor comprising a cell culture chamber;
b) placing said mammalian stem cells within said culture chamber;
c) providing a perfusion loop which provides fresh medium to said perfused bioreactor and removes used medium from said perfused bioreactor;
d) providing a dialysis loop which comprises a reservoir of medium and dialysis chamber;
   wherein the dialysis loop provides fresh medium to the perfusion loop through the dialysis chamber.

As used herein the expression “mammalian stem cells” has its general meaning in the art and designates cells of mammalian origin capable of self-renewal and capable of differentiating into a diverse range of specialized cells. A mammal can be, for example, a rodent, a feline, a canine or a primate. Preferably, a mammal according to the invention is a human.

Examples of mammalian stem cells according to the invention include both multipotent and totipotent stem cells, embryonic stem cells, gonadal stem cells, somatic stein/progenitor cells, haematopoietic stem cells, amniotic cells, epidermal stem cells and neuronal stem

Preferably, mammalian stem cells according to the invention are embryonic stem (ES) cells. Still more preferably, mammalian stem cells according to the invention are human ES (hES) cells.

As used herein, the term “culturing” can refer to the growth, proliferation and differentiation of cells into other cell types, or into multicellular bodies. When applied to ES cells, “culturing” can refer to the formation of three-dimensional aggregates called “embryoid bodies”, which can then be further differentiated into a variety of cell types.

As used herein, the term “perfused bioreactor” refers to bioreactor to which fresh culture medium is continuously added and from which used culture media is continuously removed.

In a preferred embodiment, the perfused bioreactor according to the invention is a perfused low shear stress bioreactor. Examples of low shear stress bioreactors can be found in US Application No. 2005/0095700 which describes perfusion systems comprising a cell retention device or in U.S. Pat. No. 5,308,764 which describes a Slow Turning Lateral Vessel (STLV).

In a preferred embodiment, the perfused bioreactor is a bioreactor with low shear stress environment and high mass transfer capacity by radial diffusion or gentle agitation.

In a preferred embodiment, the perfused bioreactor according to the invention is a STLV. STLVs are commercially available, for example, from Synthecon, Cellon SA, Bereldange, Luxembourg.

As used herein, the term “perfusion loop” refers to a closed circuit of medium which circulates between the perfused bioreactor of the invention and the dialysis chamber.

Typically, the perfusion loop has a flow which enables the renewal of all medium in the culture chamber between 12 and 48 hours In a preferred embodiment, the perfusion loop has a flow which enables the renewal of all medium in the culture chamber within 24 hours.

As used herein, the expressions “fresh medium” refers to the culture medium which is rich in nutrients (such as glucose and essential amino acids) and O₂ but poor in metabolic waste products (such as lactate, glutamate, ammonia) and CO₂.

As used herein, the expression “used medium” refers to the culture medium which is poor in nutrients and O₂ but rich in metabolic waste products and CO₂.

As used herein, the expression “dialysis loop” refers to a closed circuit of medium which circulates between the dialysis chamber and the reservoir of medium.

Typically, the dialysis loop has a flow which enables the renewal of all medium in the dialysis chamber between 1 and 12 hours. In a preferred embodiment, the dialysis loop has a flow which enables the renewal of all medium in the dialysis chamber within 2 hours.

The reservoir of medium according to the invention can be any recipient suitable for containing a volume of fresh medium. Examples are well-known in the art and include flasks, bottles, cylinders etc.

In a preferred embodiment, the volume of the reservoir of medium is superior to that of the culture chamber of the bioreactor. Still more preferably, the volume of the reservoir of medium is at least 10 times that of the culture chamber.

Dialysis is a well-known process in the art which enables the separation of molecules in solution by the difference in their rates of diffusion through a semi-permeable membrane. In a preferred embodiment, dialysis is carried out by tangential cross-flow filtration.

Semi-permeable membranes suitable for the method of the invention can be made out of a variety of materials, including but not limited to, cellulose, nitrocellulose and other cellulose derivatives, acetate, polytetrafluoroethylene (PTFE, also known as Teflon™), polyvinylidine difluoride (PVDF), polyethersulfone (PES), nylon etc. Such semi-permeable membranes are widely available, for example, from Whatman or Millipore.

Semi-permeable membranes are available in a variety of configurations: flat plates, tubular modules, spiral wound modules and hollow fibers.

Every semi-permeable membrane is characterized by its porosity, or molecular weight cut-off (MWCO), which determines the maximum size of the molecules which can diffuse through said semi-permeable membrane.

Typically, the semi-permeable membrane of the dialysis loop has a MWCO comprised between 10 and 25 kDa. In a preferred embodiment, the MWCO of the semi-permeable membrane of the dialysis loop is 12 kDa. In this manner, the semi-permeable membrane is permeable to small molecular weight nutrients and waste products and gases, but impermeable to higher molecular weight products such as growth factors, differentiation products, cytokines or other adjunct products.

Typically, the perfused bioreactor of the invention further comprises a semi-permeable membrane which separates the culture chamber from the circulating medium of the perfusion loop.

Typically, the semi-permeable membrane of the perfusion loop has a MWCO comprised between 100 and 500 kDa. In a preferred embodiment the MWCO of the semi-permeable membrane of the perfusion loop is 100 kDa. In this manner, the semi-permeable membrane is impermeable to the cells but enables the diffusion of the small molecular weight nutrients and waste products, gases etc.

Typically, circulation of medium within the perfusion loop is performed by using a pump. Suitable pumps according to the invention include, but are not limited to, peristaltic pumps. In a preferred embodiment, the pump is placed upstream of the perfused bioreactor.

Typically, the method of the invention further comprises providing a bubble trap.

In a preferred embodiment, the bubble trap is placed before the dialysis chamber in order to avoid pump failure.

Typically, circulation of medium within the dialysis loop is performed by using a pump.

In a preferred embodiment the method of the invention further comprises providing an oxygenator.

Suitable oxygenators according to the invention are well known in the art. Any apparatus suitable for the delivery of oxygen to the culture medium can be used. Examples are membrane oxygenators, such as silicone membrane oxygenators available from Synthecon.

In a preferred embodiment, the oxygenator of the invention is placed in the perfusion loop. Even more preferably, the oxygenator is placed upstream of the perfused bioreactor.

In a preferred embodiment, the method of the invention further comprises providing bioanalyzer.

The term “bioanalyzer” as used herein designates any apparatus suitable for measuring a variety of biophysical and biochemical parameters, such as pH, pO₂, pCO₂, osmolarity, and the concentration of small molecular weight molecules, such as glucose, glutamine, glutamate, lactate, sodium, potassium, phosphate and ammonium. A bioanalyzer according to the invention can be a HPLC column.

In a preferred embodiment, the bioanalyzer is placed downstream of the perfused bioreactor.

In a further embodiment, the invention relates to a device for culturing mammalian stem cells comprising:
a perfused low shear stress bioreactor with high mass transfer capacity comprising a cell culture chamber;
a perfusion loop connected to said perfused low shear stress bioreactor;
a dialysis loop comprising a dialysis chamber connected to said perfusion loop;
   wherein, when in use, the perfusion loop provides fresh medium to the perfused low shear stress bioreactor and removes used medium from the perfused low shear stress bioreactor; and
   wherein, when is use, the dialysis loop provides fresh medium from a reservoir of medium to the perfusion loop through the dialysis chamber.

In a preferred embodiment, the device according to the invention further comprises an oxygenator. Even more preferably, said oxygenator is placed in the perfusion loop. Still more preferably, said oxygenator is placed upstream of the perfused low shear stress bioreactor.

All the features described for the method of the invention can be advantageously incorporated into the device according to the invention. For example, the device according to the invention can further comprise pumps, a bubble trap, an oxygenator, a bioanalyzer.

The invention also relates to a system comprising a device according to the invention and mammalian stem cells placed within the culture chamber of the perfused low shear stress bioreactor.

The method of the invention advantageously allows for the long term culturing of mammalian stem cells since it enables continuous dilution of waste products over time, maintain of the concentrations of nutrients, while restricting the need for renewing the medium, thus preventing the waste of expensive adjunct products. Adjunct products are particularly important for the culture of mammalian stem cells since they will, together with the controlled aggregation of the cells into multicellular bodies, determine the viability and the fate of the cells

Moreover, the method of the invention allows changing the medium in the culture chamber, whenever needed, without altering the growth process, since the reservoir of medium is in a separate loop from the perfusion loop. This is particularly interesting for the culture of mammalian stem cells, because of the fragility of the multicellular bodies. Indeed, interruption of the stirring, and of the microgravity in STLVs, would lead to agglomeration of the multicellular bodies into large agglomerates, which would disturb their growth and differentiation.

Furthermore, the method of the invention allows for the direct analysis of the culture medium, thus enabling the precise control of the microenvironment in which the cells are growing. This is particularly important in the case of mammalian stem cells, since fine control of cell culture conditions is an absolute requisite both for the traceability of industrial processes and safety of clinical grade cell therapy products.

The invention will be further illustrated in view of the following figures and examples.

### FIGURE LEGENDS

FIG. 1—Diagram of the perfused/dialysed STLV bioreactor.

The dialysis loop (in grey) comprises the medium tank, a pump and the outer part of the dialysis chamber equipped with a semi-permeable membrane. The culture loop (in black) comprises the STLV bioreactor, a pump, an oxygenator, a bubble trap, a bioanalyser and the inner part of the dialysis chamber. Only the STLV chamber rotates continuously.

The arrow indicates the rotation of the perfused bioreactor.

Perfused bioreactor (1), culture chamber (2), reservoir of medium (3), dialysis chamber (4), semi-permeable membrane (5), semi-permeable membrane (6), pump (7), bubble trap (8), pump (9), oxygenator (10), bioanalyzer (11).

FIG. 2—Culture medium analyses between day 0 and day 4 of differentiation.

(A) Levels of glucose (Gluc, ) and lactate (Lac, ♦); (B) glutamate (Glu, ▴) and ammonium (NH4, ▪) in the culture medium in Petri dish, non-perfused STLV, perfused rotating bioreactor and perfused/dialysed STLV.

FIG. 3—OCT4 and NANOG gene expression in EBs analysed by quantitative PCR at 48 (D2) and 96 (D4) hours.

(A) Fold change in gene expression in EBs differentiated in perfused/dialysed STLV versus Petri dish (PD). (B-C). Plot box of OCT4 and NANOG expression values for individual EBs. (D) Typical gene expression levels in individual EBs.

FIG. 4—Fold change in gene expression in EBs differentiated in perfused/dialysed STLV versus Petri dish after 4 days (black bars) and 8 days (grey bars).

(A) NANOG, E Cadherin and germ layers markers, alpha-foetoprotein (AFP), brachyury (T), FGF5 and SOX1. (B) Neural markers: SOX1, PAX6, Nestin, NCAM. Means±SD (n=3).

FIG. 5—Comparison of neural progenitors generated in perfused/dialysed STLV and following co-culture with MS5 stromal cells.

(A) Diagram of neural differentiation protocols: ▾ signals the appearance of neural progenitors organized in rosettes in the culture. qPCR analysis of OCT4 and NANOG (B) and neural markers (C) normalized with reference to their levels in undifferentiated hES or in the 8 week-old human foetal brain (FB), respectively.

FIG. 5bis—Comparison of neural progenitors (NP) generated in perfused/dialyzed STLV, perfused STLV, non-perfused STLV, SCC and following co-culture with MS5 stromal cells (H9 line).

(A) Schematic diagram illustrating the sequential step of neural differentiation protocols: ▾ signals rosettes-like cells appearance (n=4). Quantitative PCR analysis of OCT-4 and NANOG (B), and of neural markers (C), respectively normalized on undifferentiated hESC and human fetal brain (P-value<0.05 for all conditions using the t-test after Welch correction). (D) Immunostaining of neural progenitors obtained following a culture step in p/dialyzed STLV (a, b), non-perfused STLV (c, d), perfused STLV (e, f), SCC (g, h) and using MS5 induction (i, j). In a, c, e, g and i, TUJ-1 and NESTIN immunoreactivity (in red and green, respectively); in b, d, f, h and j, OCT-4 and PAX-6 (in red and green, respectively). Scale bar=50 μm. (D) Immunostaining of neural progenitors obtained following a culture step in p/dialyzed STLV (a, b), non-perfused STLV (c, d), perfused STLV (e, f), SCC (g, h) and using MS5 induction (i, j). In a, c, e, g and i, TUJ-1 and NESTIN immunoreactivity (in red and green, respectively); in b, d, f, h and j, OCT-4 and PAX-6 (in red and green, respectively). Scale bar=50 μm.

FIG. 6—Progression towards three germ layers stage from SCC (♦) and p/dialyzed STLV (▪) cultures in EB (Oct-4/GFP Hues-9 line).

The determination of induction and progression of differentiation was performed by control of percentage of total population expressing the Green Fluorescent Protein (GFP) under OCT-4 full length promoter expression (dotted lines) and NCAM (dark lines) monitoring by flow cytometry over time the first five days. Data are shown as means±SD (n=3).

FIG. 7—OCT-4 gene expression in individual EBs from SA01 cell line at day 3, analyzed by quantitative PCR and normalized on hESC level. The black arrows indicate EBs with expression at the level of undifferentiated hESC level, dark bars shows means of relative expression to undifferentiated level. Indicated P-values<0.06 was performed by use of the t-test after Welch correction.

### EXAMPLE

### Material and Methods

### hES Cell Culture

The human ES cell line VUB01 (XY, passage 80), derived at the Vrije Universiteit Brussels (Mateizel et al., 2006), SA01 distributed by Cellartis (Sweden), H9 (WA09, WiCell Research Institue) and HUES-9 OCT-4GFP., in which GFP is under the control of the full length POU5F1 (OCT-4) promoter, kindly given by Chad Cowan (Harvard Stem Cell Institute), were also used during this study. Cells were maintained on a feeder layer of mitomycin C-inactivated murine STO (Sim's Thioguanine Ouabaine Resistant) fibroblasts in Knock-Out (KO)-DMEM supplemented with 20% of KO Serum Replacement (KSR), 1 mM L-glutamine, 0.1% penicillin/streptomycin, 1% non-essential amino acids and 4 ng/ml FGF2 (all from Invitrogen, Cergy, France). Culture medium was changed by half daily, supplemented by 8 ng/ml FGF2. For passaging, the cells were harvested using collagenase type IV (1 mg/ml, 5 min). The dish was washed twice with hES medium and gently scraped with a plastic pipette. The hES medium was gently aspired and transferred into a 15 ml conic tube in order to separate by passive sedimentation isolated cells (most likely STO) and small clumps of hES that were calibrated by filtration onto Cell Strainer (70 μm, Beckton Dickinson, Le-Pont-de-Claix, France). The supernatant was gently removed, and the re-suspended hES seeded at a 1:5 ratio, in 300 cm² flask.

After 7 days, each flask contained approximately 50 millions undifferentiated hES cells by flask. They were treated using 5 ml collagenase type IV and dispase I (at 1 and 0.3 mg/ml, respectively) at 37° C., 5% CO₂ for 20 min. The colonies of hES thus detached without dispersing STO. After elimination of remaining feeder cells by 100 μm filtration, hES colonies were broken into small clumps and filtered again onto a 70 μm Cell Strainer, before size control under the microscope.

Human ES cells were seeded in the bioreactor chamber at approximately 0.5 million cells per ml, in hES medium without FGF2. The bioreactor was set to rotate at 12 rpm and the speed was increased daily by 1 rpm up to a plateau at 20 rpm. The perfusion flow was set to renew all medium in the chamber within 24 hours (9 rpm). Control hEBs formed in Petri dish (static culture condition, SCC) were seeded at the same concentration to obtain the same rate of aggregation; medium was replaced by half daily.

### STLV Bioreactors, Set Up and Function

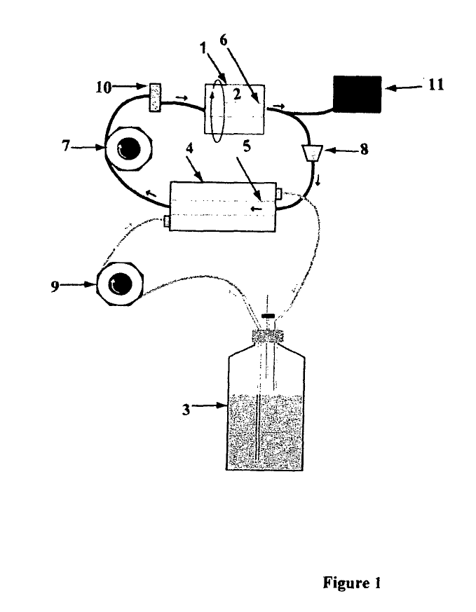

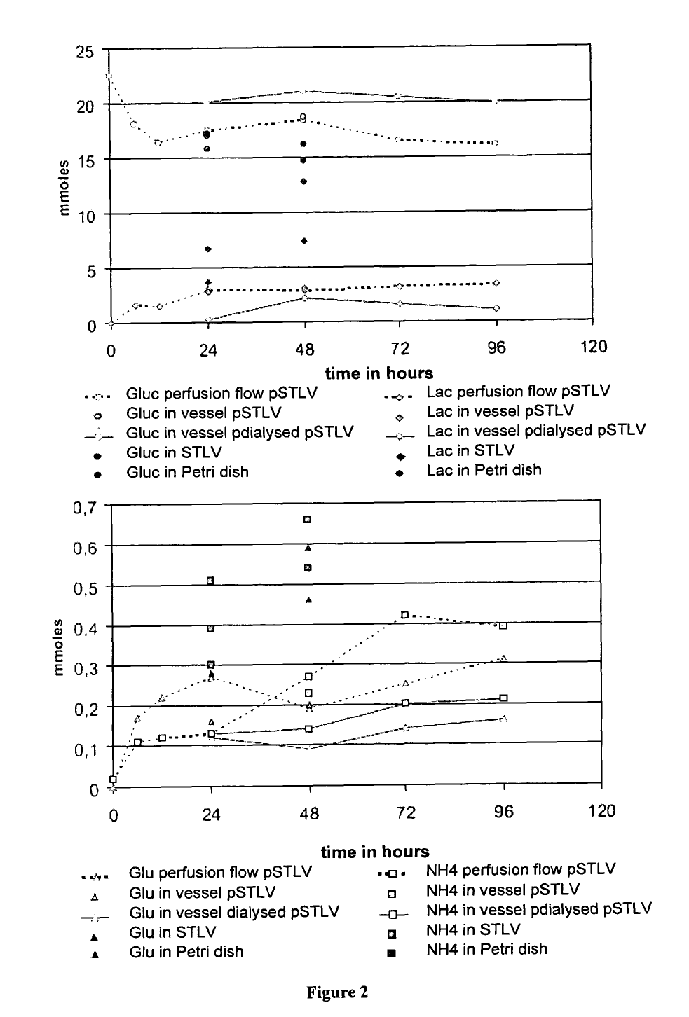

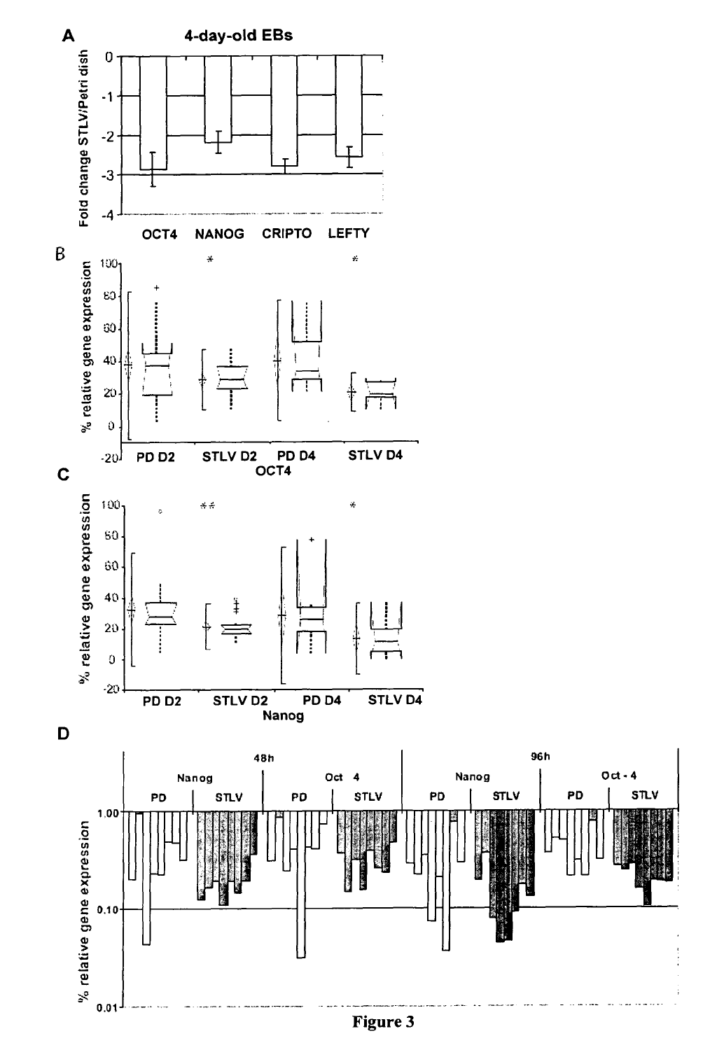

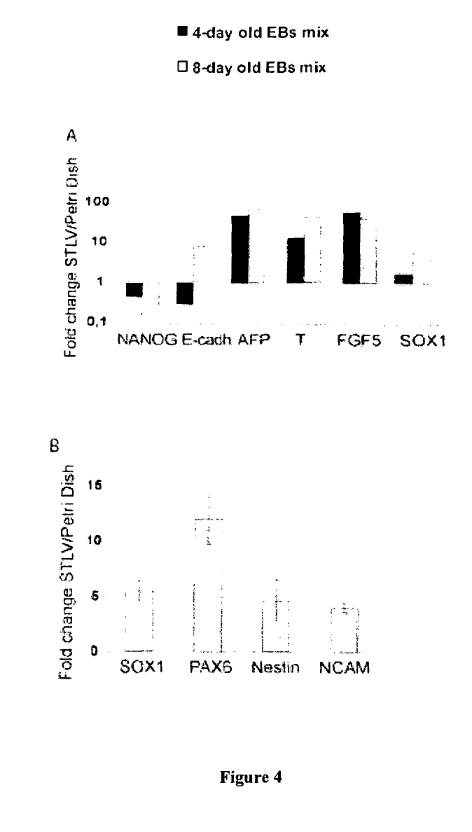

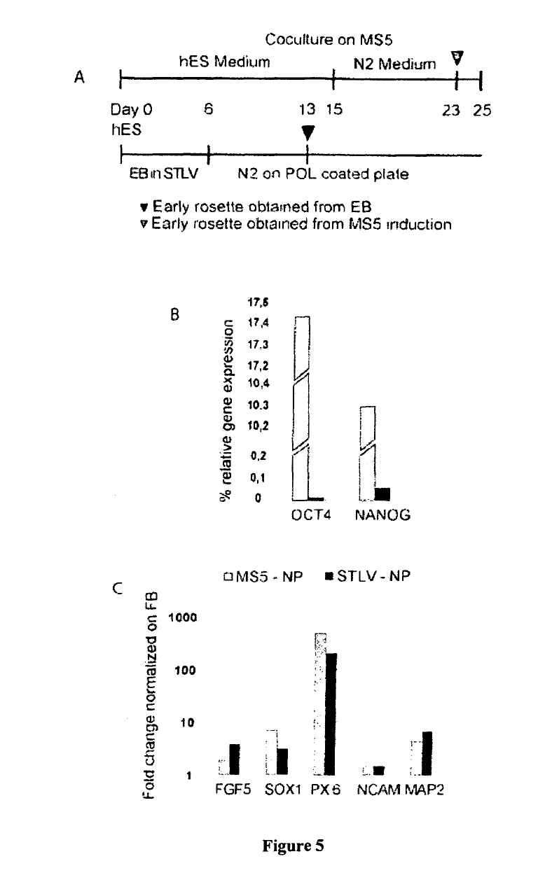

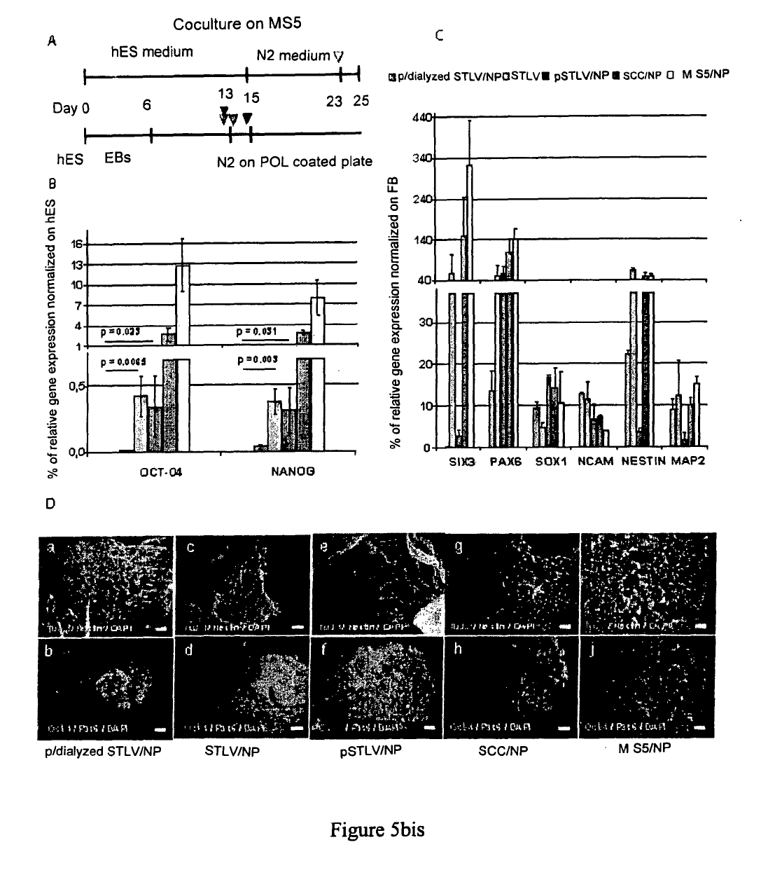

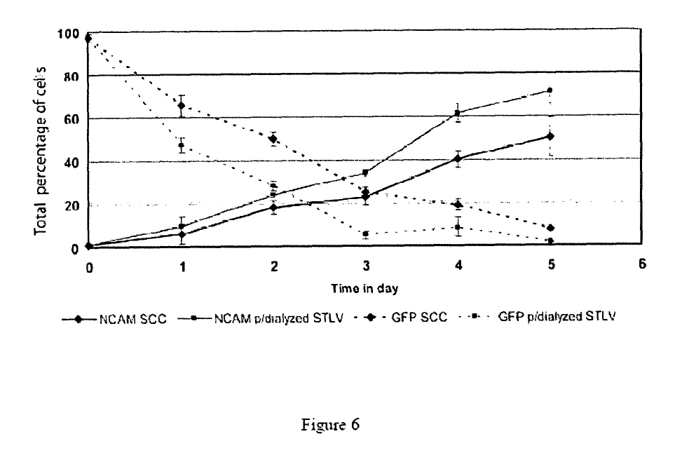

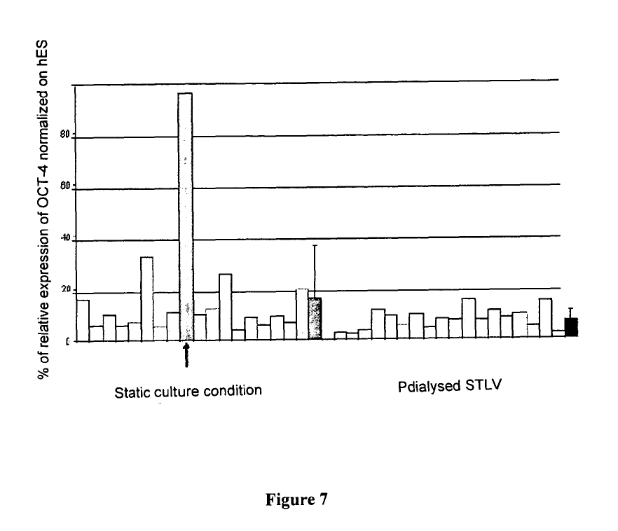

The perfused STLV bioreactor (1 in FIG. 1) included an autoclaved 55 ml-wide culture chamber (2). The system also contained variable speed motor drives with tachometers, a culture tank, a peristaltic pump (7) and a silicone membrane oxygenator (10) (all from Synthecon, Cellon SA, Bereldange, Luxembourg). All components were connected using flexible silicone tubing. The used medium outlet was covered by a dialysis membrane (6) with a 100 kD molecular weight cut-off at the inner cylinder of the perfused STLV, in order to keep cells out. The second loop consisted of a dialysis chamber (4) (200 ml) with an inner cylinder covered by a dialysis membrane (5) with a 12 kD molecular weight cut-off. A transmembrane flow was produced by a second pump (9), set to provide full medium renewal within 24 hours. A bubble trap (8) was added on the first loop to avoid pump failure.

Culture conditions were controlled online by connection of the used medium at the outlet of the STLV to a Bioprofile 400 bioanalyzer (11) (Nova Biomedical, les Ulis, France). Online analysis was performed every 6 hours for pH, pO₂, pCO₂, osmolarity, concentration of glutamine, glutamate, glucose, lactate, sodium, potassium and ammonium. The bioanalyzer (11) was programmed, according to results of preliminary experiments, to initiate calibration cycles at regular intervals every 6 hours. Additional manual calibration and quality controls were performed whenever deemed useful. Preliminary control experiments confirmed that, on the basis of those regular analyses all physicochemical parameters could be maintained stable over time in the culture chamber (Table 2).

For EBs sampling, the rotating vessels were stopper and placed on a clean bench to allow the cell aggregates to settle and take away by pipetting. Human EBs were individually retrieved from either the culture chamber (2) of the STLV bioreactor (1) or control Petri dish SCC after 2, 4 and 8 days. Mix samples were also retrieved on the same days. For retrieval from the STLV culture chamber (2), 500 μl of medium were retrieved and the hEBs filtered out using a 70 μm-pore nylon cell strainer (BD). The strainer was rinsed with PBS in order to deliver the hEBs to a Petri dish. Resulting aggregates were individually collected under a stereomicroscope, and placed each in 100 μl of RLT lysis buffer (Qiagen, Courtaboeuf, France). Mix samples were formed by the remaining, non-individually collected filtered aggregates; they were centrifuged (900 rpm, 1 minute) and collected in 1 ml RLT lysis buffer.

### Neural Differentiation Protocols

Differentiation of the H9 cell line along the neural lineage was performed by two different manners.

First, hEBs produced in SCC or p/dialyzed STLV were plated after 6 days of aggregation. They were transferred onto polyornithine/laminin-coated (POL) culture dishes in DMEM/F12 supplemented with N2 medium that was replaced every 2-3 days. Morphologically identified neural rosettes were isolated mechanically whenever they appeared.

Second, neural progenitors were obtained from undifferentiated hES by co-culture with MS5 stromal cells, as described previously (Perrier et al., 2004). Briefly, hES cells were plated at 0.2-1×10³ cells per cm² on a confluent MS5 layer inactivated by treatment with mitomycin C, in serum replacement medium containing DMEM, 20% KSR, 2 mM I-glutamine and 10 μM β-mercaptoethanol. After 15 days, cultures were switched to DMEM/F12-N2 medium. Medium was changed every 2-3 days, and morphologically identified neural rosettes were isolated mechanically from feeders.

### Real-Time RT-PCR

Total RNA was isolated from hES (undifferentiated cells) and hEBs (differentiated cells) using RNeasy extraction kits (Qiagen). Cells were lysed in RLT buffer before homogenizing the lysates with 1 volume of 70% ethanol. Samples were applied to an RNeasy minElute Spin Column. On-column DNase I digestion was carried out during the isolation using an RNase-free DNase set (Qiagen). RNA bound to the silica-gel membrane was finally eluted in RNase-free water. After quantitative measure of RNA using the Nanodrop technology, reverse transcription was performed using the Superscript II reverse transcription kit (Invitrogen).

For all samples, including isolated hEBs, real-time RT-PCR was performed using a Chromo4 real time system (Bio-Rad, Marne la Coquette, France) and SYBR Green PCR Master Mix (Applied Biosystem, Courtaboeuf, France) following the manufacturer's instructions. Quantification of gene expression was based on the Ct (Cycle threshold) value calculated using the Opticon Monitor software. Melting curve and electrophoresis analyses were performed to control PCR products specificities and exclude non-specific amplification. The PCR Primers are listed in Table 1. The annealing temperature of all the primers was 60° C. Samples were normalized against βTubulin. Experiments were normalized with reference to undifferentiated hESC or human foetal brain (FB, Ozyme, Saint-Quentin-en-Yvelines, France).

### Fluorescence-Activated Cell Sorting (FACS) Analysis

Hues-9 POU5F1/GFP EBs obtained in SCC or p/dialyzed STLV were enzymatically dissociated with Tryple™ Select (Invitrogen) for 15 min at 37° C., washed and resuspended in 1 ml FACS buffer (2% FBS, in PBS). Cells were probed for 30 min at 4° C. with monoclonal r-phycoerythrin-mouse anti-human CD 56 (N-CAM) Clone B159 or r-phycoerythrin-isotype control (R&D, France). Stained cells were then analyzed in duplicate on a FACScalibur flow cytometer using CellQuest software (BD Biosciences, France).

### Immunocytochemistry

Cells were fixed with 4% paraformaldehyde for 10 min at room temperature before blocking and permeabilizing with PBS 2%, Bovine Serum Albumin, 0.1% Triton X-100. Primary antibodies were incubated overnight at 4° C. in blocking buffer, included rabbit polyclonal antibodies raised against PAX6 (Covance), mouse monoclonal antibodies raised against OCT-4 (Chemicon), N-CAM clone Eric1 (Santa Cruz) and class III-tubulin (Tuj1, Covance). Cells were then stained with the appropriate fluorophore-conjugated secondary antibody and DAPI.

### Statistical Analyses

All data analyses, including graphical representations, were performed using Microsoft Excel and Analyse-it General statistics (Analyse-it Software, Ltd. Leeds, UK). Values are presented as means±standard deviation. A Fisher test was conducted to determine variance equality between samples. The statistical significance level of a difference between samples was determined using Student's t-test when the Fisher test was positive, Welch t-test was applied (indicated in the text by Welch's approximation) when it was not.

TABLE 1

PCR primers

primer sequences

| | | |
|---|---|---|
| gene | Forward | Reverse |

| | | |
|---|---|---|
| bTUB | ATCAGCAAGATCCGGGAAGAG | CCGTGTCTGACACCTTGGGT |
| | (SEQ ID NO. 1) | (SEQ ID NO. 2) |
| HPRT1 | ATGGGAGGCCATCACATTGT | ATGTAATCCAGCAGGTCAGCAA |
| | (SEQ ID NO. 3) | (SEQ ID NO. 4) |
| L13a | CCTGGAGGAGAAGAGGAAAGAGA | TTGAGGACCTCTGTGTATTTGTCAA |
| | (SEQ ID NO. 5) | (SEQ ID NO. 6) |
| UbC | ATTTGGGTCGCGGTTCTTG | TGCCTTGACATTCTCGATGGT |
| | (SEQ ID NO. 7) | (SEQ ID NO. 8) |
| GAPDH | CCCACTAACATCAAATGGGG | CCTTCCACAATGCCAAAGTT |
| | (SEQ ID NO. 9) | (SEQ ID NO. 10) |
| bACTIN | CTCTTCCAGCCTTCCTTCCT | AGCACTGTGTTGGCGTACAG |
| | (SEQ ID NO. 11) | (SEQ ID NO. 12) |
| OCT4 | CTTGCTGCAGAAGTGGGTGGAGGAA | CTGCAGTGTGGGTTTCGGGCA |
| | (SEQ ID NO. 13) | (SEQ ID NO. 14) |
| NANOG | CAAAGGCAAACAACCCACTT | TCTGCTGGAGGCTGAGGTAT |
| | (SEQ ID NO. 15) | (SEQ ID NO. 16) |
| LEFTYA | GGGAATTGGGATACCTGGATTC | TAAATATGCACGGGCAAGGCTC |
| | (SEQ ID NO. 17) | (SEQ ID NO. 18) |
| AFP | ACTGCAATTGAGAAACCCACTGGAGATG | CGATGCTGGAGTGGGCTTTTTGTGT |
| | (SEQ ID NO. 19) | (SEQ ID NO. 20) |
| CRIPTO | ACAGAACCTGCTGCCTGAAT | ATCACAGCCGGGTAGAAATG |
| | (SEQ ID NO. 21) | (SEQ ID NO. 22) |
| SOX1 | GATGCACAACTCGGAGATCA | GTCCTTCTTGAGCAGCGTCT |
| | (SEQ ID NO. 23) | (SEQ ID NO. 24) |
| PAX6 | GCCAGCAACACACCTAGTCA | TGTGAGGGCTGTGTCTGTTC |
| | (SEQ ID NO. 25) | (SEQ ID NO. 26) |
| FGF5 | TGCAAGTGCCAAGTTCACAGA | AGTTCTATGTATTGCTGAGGCATAGGTA |
| | (SEQ ID NO. 27) | (SEQ ID NO. 28) |
| BRACHYURY | ATCACCAGCCACTGCTTC | GGGTTCCTCCATCATCTCTT |
| | (SEQ ID NO. 29) | (SEQ ID NO. 30) |
| E-CADHERIN | AGGAATTCTTGCTTTGCTAATTCTG | CGAAGAAACAGCAAGAGCAGC |
| | (SEQ ID NO. 31) | (SEQ ID NO. 32) |
| NESTIN | GGAAGAGAACCTGGGAAAGG | CTTGGTCCTTCTCCACCGTA |
| | (SEQ ID NO. 33) | (SEQ ID NO. 34) |

### Results

### The Perfused/Dialysed Rotating Bioreactor System

The perfused/dialysed STLV is constituted of two loops of medium perfusion. The first cell perfusion loop feeds the bioreactor. Protein complementation of the medium is performed at this level. The perfusate goes though the STLV chamber equipped with a semi-permeable membrane and an oxygenator. For online control of culture condition, an automated bioanalyzer (Bioprofile 400) is connected at the outlet and allows non-invasive follow-up of the culture parameters. In addition a bubble trap is installed to prevent pump failure while retrieving medium for analysis. The dialysis loop contains a large medium tank connected to a dialysis chamber, the function of which is to dilute the dyalisate with fresh medium. It enhances waste elimination and nutrient supply by exchange between the two compartments of the dialysis chamber through a semi-permeable membrane with a 12 kDa cut-off. The flow of the two perfusion loops is controlled in an independent way by two pumps to provide full renewal within 24 hours. (FIG. 1).

To validate the effectiveness of this system we measured the main physicochemical parameters over ten days of EBs culture (Table 2), namely pH, partial pressure of CO₂ and O₂, osmolarity.

All these parameters are indirect indicators of homeostasis, glucid, respiratory and ionic metabolism, respectively, and together reveal the mass transfer capacity of the system. The results obtained indicated a robust stability for all parameter with values near to those of fresh medium.

TABLE 2
Variations over time in the culture medium in the
culture loop of the system described in FIG. 1.

| | Mean | SD |
|---|---|---|
| pH | 7.27 | 0.025 |
| pCO2 mm Hg | 33.7 | 1.6 |
| pO2 mm Hg | 129.7 | 10.4 |
| mOsm/kg | 334 | 3.4 |

Four key physicochemical parameters were measured between day 0 and day 10, twice a day. Variations of the physical parameters are presented as standard deviation (SD) of the means.

### Comparison of Biological Parameters in the Culture Medium Over Time in Petri Dish, Non-Perfused, Perfused and Perfused/Dialysed STLV

FIG. 2 summarizes the results of successive analyzes in the culture medium of concentrations for glucose, glutamate, NH₄ and lactate. Analyzes could be carried out over only 2 days in Petri dish and non-perfused STLV because accumulating cell waste imposed changing the medium.

Results obtained in Petri dish and non-perfused STLV were similar, showing a progressive 30% decrease in glucose concentration and a rapid parallel increase in cell waste, with lactate reaching close to 13 mmoles and NH₄ doubling over the second day of culture up to 0.65 mmoles.

In sharp contrast, addition of the continuous perfusion to the STLV stabilized concentrations over time at levels well compatible with cell culture. Glucose concentration decreased by 30% as in Petri dish and non-perfused STLV during the first 12 hours of aggregation, but then plateaued up to four days at similar levels, despite continuous cell expansion in the chamber. All parameters indicative of cell waste increased in the medium over the first 24 hours. Glutamate and lactate then remained stable up to 4 days, at 4 and 0.2 mmoles respectively. NH4 doubled again within 48 hours to reach a plateau at 0.4 mmole, i.e. a level still compatible with cell maintenance.

Addition of a dialysis chamber (4) to the perfused STLV (1), to generate the method of the invention, systematically improved further all values. Glucose concentration remained around 20 mmoles up to 4 days, i.e. increased by about 25% as compared to the non-dialysed system. In parallel, all indicators of cell waste decreased 2-fold as compared to the non-dialysed system. Lactate was in the mmolar range at all times, glutamate and NH4 around 0.2 mmole.

The best results were obtained with the method of the invention, i.e, the perfused/dialysed STLV. All subsequent analyses were based on this method for culturing hES cells.

Comparison of the Differentiation of Human Embryoid Bodies Over Time in Petri Dish and Perfused/Dialysed STLV

Two main features of the differentiation of hEBs were analyzed in order to compare results obtained using classical “Petri dish” techniques and the perfused/dialysed STLV, namely the decrease in expression of markers of the undifferentiated stage, and the increase in expression of markers of differentiation. For undifferentiation, we chose NANOG and OCT4, two transcription factors associated with the undifferentiated hES stage, and added CRIPTO and LEFTY that participate specifically in this stage. To analyze differentiation, we selected two types of genes, early markers of the three embryonic layers, alphafetoprotein (AFP), brachyury (T), FGF5 and SOX1, and markers of a later stage of neural differentiation, PAX6, NESTIN and CD56/N-CAM. In addition, we determined the course of expression of E-cadherin as it is known to peak during the phase of aggregation, then is down-regulated before increasing again at gastrulation (Dang et al., 2005). This specific time course of expression was used to mark both the effectiveness of aggregation and the completeness of gastrulation.

Comparison of mixes of 4 days-old hEBS showed that all markers of undifferentiation were decreased two times (Fold-change≈2, P value<0.01 n=3) more in the perfused/dialysed STLV than in Petri dish (FIG. 3A), in a similar way for transcription factors and other markers.

Those results were confirmed by FACS analysis on cells expressing GFP under the control of the promoter of OCT-4, which showed a 24 hours delay in the differentiation kinetics at the earliest time-points for the p/dialyzed STLV conditions as compared to SCC (FIG. 6).

In the analysis of individual hEBs, similarly, expression of OCT4 and NANOG decreased more rapidly in perfused/dialysed STLV than in Petri dish, in a statistically significant manner at 48 and 96 hours (FIGS. 3B,C; Student t-test, *: <0.05; **: <0.01). This apparent enhancement of the down-regulation of undifferentiation markers in the perfused/dialysed STLV was associated with a more homogeneous differentiation of the hEBs (FIG. 3D). Human EBs in Petri dish indeed showed very variable results, including cases with defective aggregation and persistency of an undifferentiated gene pattern of expression.

These observations were confirmed with another cell line, SA-01 (FIG. 7).

Analysis of differentiation markers in mix samples of hEBs at 4 and 8 days confirmed the overall acceleration of the differentiation. At 4 days, E-cadherin expression was 5 times lower in STLV-derived hEBs than in static conditions. At 8 days, the reverse was observed with E-cadherin expressed 8 times more in the former than in the latter, indicating a faster progression toward gastrulation. Accordingly, changes in gene expression were 10 to 50 fold for AFP, brachyury (T) and FGF5 at both time points, 6 fold for SOX1 at 8 days (FIG. 4A). At that moment, expression of genes associated with later stages of neural differentiation was also increased 5 to 10 times more in the perfused/dialysed STLV than in Petri dish (FIG. 4B).

Comparison of Neural Differentiation of hEBs Produced in the Perfused/Dialysed STLV and Using Co-Culture with Stromal Cells

### Experiment 1

Using co-culture with MS5 feeder cells, early neural rosettes were observed 23 days after beginning of the induction, in agreement with previously published data (Perrier et al., 2004). The process was much faster using the perfused/dialysed STLV to grow hEBS as early neural rosettes were then collected after only 13 days (6 days in the bioreactor and 7 days after plating hEBs (FIG. 5A). Furthermore, real time PCR for the markers of undifferentiation OCT4 and NANOG showed virtually no expression in the STLV-derived rosettes whereas both remained expressed in co-culture-derived rosettes at up to 17% and 10% of hES levels, respectively (FIG. 5B).

When normalized using 8 week-old foetal brain as a control, neural rosettes derived from STLV-produced hEBs demonstrated levels of expression for all neural marker genes tested (FGF5, SOX1, PAX6, NCAM) similar to those observed in neural rosettes derived by co-culture (FIG. 5C).

### Experiment 2

To analyze the effect of rotary bioreactor on the specific neural differentiation, we have compared this differentiation for hEBs derived from H9 hESC line in the p/dialyzed STLV, perfused STLV, non-perfused STLV, static culture conditions and using co-culture with stromal cells.

As shown in FIG. 5bisA, the time delay to “neural rosette” formation grown was significantly shorter in all three STLV conditions as compared to SSC (1 to 2 days), and all were more than a week shorter than following induction of stromal cells. In STLV conditions, they were collected after only 13-14 days (6 days in the bioreactor and 7-8 days after plating hEBs) whereas early neural rosettes were only observed after 23 days using co-culture with MS5 feeder cells, in agreement with previous data (10).

Undifferentiated leftover cells in neural rosettes were analyzed by real time PCR of OCT-4 and NANOG. The expression of these markers of the undifferentiated stage was dramatically reduced STLV and perfused STLV until no detectable in the p/dialyzed STLV-derived rosettes, whereas both remained expressed in co-culture- and SCC-derived rosettes, at decreased though significant levels to the undifferentiated stage (FIG. 5bis B). All others markers of differentiation increased as expected in all three conditions. Once normalized on gene expression recorded in the brain of an 21-41 week-old human fetus, results were not significantly different for SOX-1, N-CAM, Nestin and MAP 2 (FIG. 5bis C). SIX3, a precocious and transient marker gene of neural specification, appeared down-regulated only in perfused and p/dialysed STLV conditions. The typical marker of early (rosette-associated) neural precursors, PAX6, was less expressed in in p/dialyzed STLV than in all other conditions, a result confirmed by immunocytochemical staining of cells in culture (FIG. 5bis D). In contrast, the cells expressing the marker of more advanced neuronal progenitors TUJ-1 were more numerous in p/dialyzed STLV culture than in any other condition.

### REFERENCES

All references cited are incorporated herein by reference.
Dang S M, Zandstra P W. Scalable production of embryonic stem cell-derived cells. Methods Mol Biol. 2005; 290:353-364.
Gerecht-Nir S, Cohen S, Itskovitz-Eldor J. Bioreactor cultivation enhances the efficiency of human embryoid body (hEB) formation and differentiation. Biotechnol Bioeng. Jun. 5, 2004; 86(5):493-502.
Gorba T, Allsopp T E. Pharmacological potential of embryonic stem cells. Pharmacol Res. April 2003; 47(4):269-278.
Itsykson P, Ilouz N, Turetsky T, et al. Derivation of neural precursors from human embryonic stem cells in the presence of noggin. Mol Cell Neurosci. September 2005; 30(1):24-36.
Mateizel I, De Temmerman N, Ullmann U, et al. Derivation of human embryonic stem cell lines from embryos obtained after IVF and after PGD for monogenic disorders. Hum Reprod. February 2006; 21(2):503-511.
Mitjavila-Garcia M T, Simonin C, Peschanski M. Embryonic stem cells: meeting the needs for cell therapy. Adv Drug Deliv Rev. Dec. 12, 2005; 57(13):1935-1943.
McNeish J. Embryonic stem cells in drug discovery. Nat Rev Drug Discov. January 2004; 3(1):70-80.
Perrier A L, Tabar V, Barberi T, et al. Derivation of midbrain dopamine neurons from human embryonic stem cells. Proc Natl Acad Sci USA. Aug. 24, 2004; 101(34):12543-12548.
Tian X, Morris J K, Linehan J L, Kaufman D S. Cytokine requirements differ for stroma and embryoid body-mediated hematopoiesis from human embryonic stem cells. Exp Hematol. October 2004; 32(10):1000-1009.

1. A method for culturing mammalian stem cells comprising the following steps:
a) providing a perfused bioreactor (1) comprising a cell culture chamber (2);
b) placing said mammalian stem cells within said culture chamber (2);
c) providing a perfusion loop which provides fresh medium to said perfused bioreactor (1) and removes used medium from said perfused bioreactor (1);
d) providing a dialysis loop which comprises a reservoir of medium (3) and dialysis chamber (4);
wherein the dialysis loop provides fresh medium to the perfusion loop through the dialysis chamber (4).

2. A method according to claim 1 wherein said mammalian stem cells are embryonic stem (ES) cells.

3. A method according to claim 1 wherein said mammalian stem cells are human ES (hES) cells.

4. A method according to claim 1 wherein said perfused bioreactor (1) is a perfused low shear stress bioreactor.

5. A method according to claim 1 wherein said perfused bioreactor (1) is a perfused bioreactor with low shear stress environment and high mass transfer capacity.

6. A method according to claim 1 wherein the volume of said reservoir of medium (3) is superior to that of said culture chamber (2).

7. A method according to claim 1 wherein the volume of said reservoir of medium (3) is at least 10 times that of said culture chamber (2).

8. A method according to claim 1 wherein said dialysis is carried out by tangential cross-flow filtration.

9. A method according to claim 1 wherein the MWCO of the semi-permeable membrane of the dialysis loop (5) is 12 kDa.

10. A method according to claim 1 wherein the MWCO of the semi-permeable membrane of the perfusion loop (6) is 100 kDa.

11. A method according to claim 1 further comprising providing an oxygenator (10).

12. A method according to claim 1 further comprising providing an oxygenator (10) and wherein said oxygenator (10) is placed in said perfusion loop.

13. A method according to claim 1 further comprising providing an oxygenator (10) and wherein said oxygenator (10) is placed upstream of said perfused bioreactor (1).

14. A method according to claim 1 further comprising providing a bioanalyzer (11).

15. A method according to claim 1 further comprising providing a bioanalyzer (11) and wherein said bioanalyzer (11) is placed downstream of said perfused bioreactor (1).

16. A device for culturing mammalian stem cells comprising:
a perfused low shear stress bioreactor (1) with high mass transfer capacity comprising a cell culture chamber (2);
a perfusion loop connected to said perfused low shear stress bioreactor (1);
a dialysis loop comprising a dialysis chamber (4) connected to said perfusion loop;
wherein, when in use, the perfusion loop provides fresh medium to the perfused low shear stress bioreactor (1) and removes used medium from the perfused low shear stress bioreactor (1); and
wherein, when is use, the dialysis loop provides fresh medium from a reservoir of medium (3) to the perfusion loop through the dialysis chamber (4).

17. A device according to claim 16 further comprising an oxygenator (10).

18. A device according to claim 16 wherein, when in use, said oxygenator (10) is placed upstream of said perfused low shear stress bioreactor (1) with high mass transfer capacity.

19. A system comprising a device according to claim 16 and mammalian stem cells placed within said culture chamber (2) of said perfused low shear stress bioreactor (1) with high mass transfer capacity.


SEQUENCE LISTING (34 sequences)

SEQ ID NO: 1 - DNA, 21 bp, Artificial Sequence
  Primer
atcagcaagatccgggaagag

SEQ ID NO: 2 - DNA, 20 bp, Artificial Sequence
  Primer
ccgtgtctgacaccttgggt

SEQ ID NO: 3 - DNA, 20 bp, Artificial Sequence
  Primer
atgggaggccatcacattgt

SEQ ID NO: 4 - DNA, 22 bp, Artificial Sequence
  Primer
atgtaatccagcaggtcagcaa

SEQ ID NO: 5 - DNA, 23 bp, Artificial Sequence
  Primer
cctggaggagaagaggaaagaga

SEQ ID NO: 6 - DNA, 25 bp, Artificial Sequence
  Primer
ttgaggacctctgtgtatttgtcaa

SEQ ID NO: 7 - DNA, 19 bp, Artificial Sequence
  Primer
atttgggtcgcggttcttg

SEQ ID NO: 8 - DNA, 21 bp, Artificial Sequence
  Primer
tgccttgacattctcgatggt

SEQ ID NO: 9 - DNA, 20 bp, Artificial Sequence
  Primer
cccactaacatcaaatgggg

SEQ ID NO: 10 - DNA, 20 bp, Artificial Sequence
  Primer
ccttccacaatgccaaagtt

SEQ ID NO: 11 - DNA, 20 bp, Artificial Sequence
  Primer
ctcttccagccttccttcct

SEQ ID NO: 12 - DNA, 20 bp, Artificial Sequence
  Primer
agcactgtgttggcgtacag

SEQ ID NO: 13 - DNA, 25 bp, Artificial Sequence
  Primer
cttgctgcagaagtgggtggaggaa

SEQ ID NO: 14 - DNA, 21 bp, Artificial Sequence
  Primer
ctgcagtgtgggtttcgggca

SEQ ID NO: 15 - DNA, 20 bp, Artificial Sequence
  Primer
caaaggcaaacaacccactt

SEQ ID NO: 16 - DNA, 20 bp, Artificial Sequence
  Primer
tctgctggaggctgaggtat

SEQ ID NO: 17 - DNA, 22 bp, Artificial Sequence
  Primer
gggaattgggatacctggattc

SEQ ID NO: 18 - DNA, 22 bp, Artificial Sequence
  Primer
taaatatgcacgggcaaggctc

SEQ ID NO: 19 - DNA, 28 bp, Artificial Sequence
  Primer
actgcaattgagaaacccactggagatg

SEQ ID NO: 20 - DNA, 25 bp, artificial
  primer
cgatgctggagtgggctttttgtgt

SEQ ID NO: 21 - DNA, 20 bp, Artificial Sequence
  Primer
acagaacctgctgcctgaat

SEQ ID NO: 22 - DNA, 20 bp, Artificial Sequence
  Primer
atcacagccgggtagaaatg

SEQ ID NO: 23 - DNA, 20 bp, Artificial Sequence
  primer
gatgcacaactcggagatca

SEQ ID NO: 24 - DNA, 20 bp, Artificial Sequence
  Primer
gtccttcttgagcagcgtct

SEQ ID NO: 25 - DNA, 20 bp, Artificial Sequence
  Primer
gccagcaacacacctagtca

SEQ ID NO: 26 - DNA, 20 bp, artificial
  primer
tgtgagggctgtgtctgttc

SEQ ID NO: 27 - DNA, 21 bp, artificial
  primer
tgcaagtgccaagttcacaga

SEQ ID NO: 28 - DNA, 28 bp, Artificial Sequence
  Primer
agttctatgtattgctgaggcataggta

SEQ ID NO: 29 - DNA, 18 bp, Artificial Sequence
  Primer
atcaccagccactgcttc

SEQ ID NO: 30 - DNA, 20 bp, Artificial Sequence
  Primer
gggttcctccatcatctctt

SEQ ID NO: 31 - DNA, 25 bp, Artificial Sequence
  Primer
aggaattcttgctttgctaattctg

SEQ ID NO: 32 - DNA, 21 bp, Artificial Sequence
  Primer
cgaagaaacagcaagagcagc

SEQ ID NO: 33 - DNA, 20 bp, Artificial Sequence
  Primer
ggaagagaacctgggaaagg

SEQ ID NO: 34 - DNA, 20 bp, Artificial Sequence
  Primer
cttggtccttctccaccgta